(19) United States
(12) Patent Application Publication
(10) Pub. No.: US 2020/0046419 A1
(43) Pub. Date: Feb. 13, 2020

(54) ELECTRIC SEALER WITH CUTTER

(71) Applicant: XCELLANCE MEDICAL TECHNOLOGIES PVT LTD, Navi Mumbai (IN)

(72) Inventor: Pradip Barsu NARKHEDE, Navi Mumbai (IN)

(21) Appl. No.: 16/343,025

(22) PCT Filed: Oct. 15, 2017
(86) PCT No.: PCT/IN2017/050473

§ 371 Date: Apr. 18, 2019

(30) Foreign Application Priority Data
Oct. 24, 2016 (IN) 201621036356

(51) Int. Cl. A61B 18/14; A61B 18/12

(52) U.S. Cl. CPC A61B 18/1445; A61B 2018/00922; A61B 18/1206

(57) ABSTRACT

A bipolar surgical device having a jaw insert (7) having jaws (4) connected to an outer tube (3) is disclosed. The jaw insert (7) and said outer tube (3) are connected to a handle (2) having at least one enclosed part (8) operating as a jaw driving mechanism and operatively coupled to a trigger (1). The jaws (4) close when said trigger (1) is pressed and said jaws (4) open when said trigger (1) is not pressed. The handle (2) consists of two buttons (6, 7′) where a first button (6) is used for sealing tissue and/or vessel when pressed, and said second button (7′) is used for cutting of the tissue and/or the vessel when pressed. The bipolar surgical device that consists of jaws comprising of sealer with cutter that is capable of sealing and/or cutting of the tissue and/or vessel.

### FIELD OF THE INVENTION

The present invention relates, in general to a surgical device and more particularly, to a surgical device having a jaw insert, outer tube, and handle which are detachable and reusable and/or disposable, the jaws seal the vessels/tissue and the cutter cuts the vessel/tissue electrically.

### BACKGROUND

Numerous bipolar electrosurgical forceps have been proposed in the past for various surgical procedures. Many of these instruments include blade members or shearing members which simply cut tissue in a mechanical and/or electromechanical manner and are relatively ineffective for vessel sealing purposes.

Other instruments rely on clamping alone to procure proper sealing thickness and are not designed to take into account gap tolerances and flatness requirements which are parameters which, if properly controlled, can assure a consistent and effective tissue seal.

In mechanical cutters, the cutting achieved is not sharp and clean, the blade can become distorted/blunt after multiple use, disposable parts are more in the device.

In electrically used cutter less force is used for cutting allowing smoother cutting, mechanical complications are less assembly wise which is simplified and device life increases.

Further, in existing devices, jaws do not allow vapors/steam generated in the sealing process to pass which decreases seal strength.

In spite of available devices and techniques, there still exists a dire need to provide a bipolar surgical device that consists of jaws comprising of sealer with cutter that is capable of sealing and/or cutting of the tissue and/or vessel.

### SUMMARY OF THE INVENTION

Accordingly, the following invention illustrates a bipolar surgical device that consists of jaws comprising of sealer with cutter that is capable of sealing and/or cutting of the tissue and/or vessel.

An aspect of the present disclosure relates to a bipolar surgical device having a jaw insert (7) having jaws (4) connected to an outer tube (3). The jaw insert (7) and said outer tube (3) are connected to a handle (2) having at least one enclosed part (8) operating as a jaw driving mechanism and operatively coupled to a trigger (1). The jaws (4) closed when said trigger (1) is pressed and said jaws (4) open when said trigger (1) is not pressed. The handle (2) consists of two buttons (6, 7′) where a first button (6) is used for sealing tissue and/or vessel when pressed, and said second button (7′) is used for cutting of the tissue and/or the vessel when pressed.

In an aspect, the jaw insert can be cleaned and/or autoclaved after it is disassembled from the outer tube or it can be replaced.

In an aspect, the outer tube can be disassembled and can be cleaned and/or autoclaved after it is disassembled and can be reused or it can be disposed.

In an aspect, said handle (2) includes a first contact pole (9), a second contact pole (10) and a third contact pole (11). In another aspect, the first contact pole (9) causes an electrical conduction on a first insert contact (18) of the jaw insert (7), the second contact pole (10) causes an electrical conduction on a second insert contact (17) of the jaw insert (7), and the third contact pole (11) causes an electrical conduction on the third insert contact (16) of the jaw insert (7). In yet another aspect, the first contact pole (9) and the second contact pole (10) causes sealing of the tissue and/or vessel while the third contact pole (11) causes the cutting effect. In still another aspect, said first contact pole (9), said second contact pole (10) and said third contact pole (11) are separated by insulators (15)

In an aspect, said handle (2) includes threaded joint (23) in which said outer tube (3) and said jaw insert (7) are detachably coupled to said handle (2).

In an aspect, said jaws (4) comprises an upper jaw (19) and a lower jaw (20) separated by spacers (22) allowing space between said upper jaw (19) and said lower jaw (20) when the jaws (4) are closed, wherein said upper jaw (19) and said lower jaw (20) are used for sealing of the tissue and/or vessel.

In an aspect, said upper jaw (19) or said lower jaw (20) includes a cutter (21) used for cutting the tissue and/or vessel.

### DESCRIPTION OF THE DRAWING

FIG. 01 illustrates the front side isometric view of the surgical device.

FIG. 02 illustrates the isometric view of the surgical device.

FIG. 03 illustrates the front view of the surgical device.

FIG. 04 illustrates the outer tube, jaw insert and handle.

FIG. 05 illustrates the sectional view displaying the internal parts of the device.

FIG. 06 illustrates the first insert contact, second insert contact and third insert contact.

FIG. 07 illustrates the upper jaw and the lower jaw that are used for sealing of the tissue and/or vessel while the cutter is used for cutting.

FIG. 08 illustrates the circuit.

FIG. 09 illustrates a cutter surrounded by insulation to isolate it from the lower jaw.

FIG. 10 illustrates a cutter with an insulation split in two halves.

FIG. 11 illustrates jaws having serrations to have better grip over a tissue.

FIG. 12 illustrates a Cross section view of jaw assembly having minimized wall thickness and increased surface area to dissipate heat faster which directly reduces the thermal spread on the tissue.

FIG. 13 illustrates a plurality of holes provided on upper jaw in order to release trapped vapors generated during sealing or cutting process.

### DESCRIPTION

The following invention illustrates a bipolar surgical device that consists of jaw assembly comprising of sealer jaws with cutter that is capable of sealing and/or cutting of the tissue and/or vessel.

FIG. 01 illustrates the front side isometric view of the surgical device displaying the handle 2, a trigger 1, outer tube 3 and jaws 4 of the instrument.

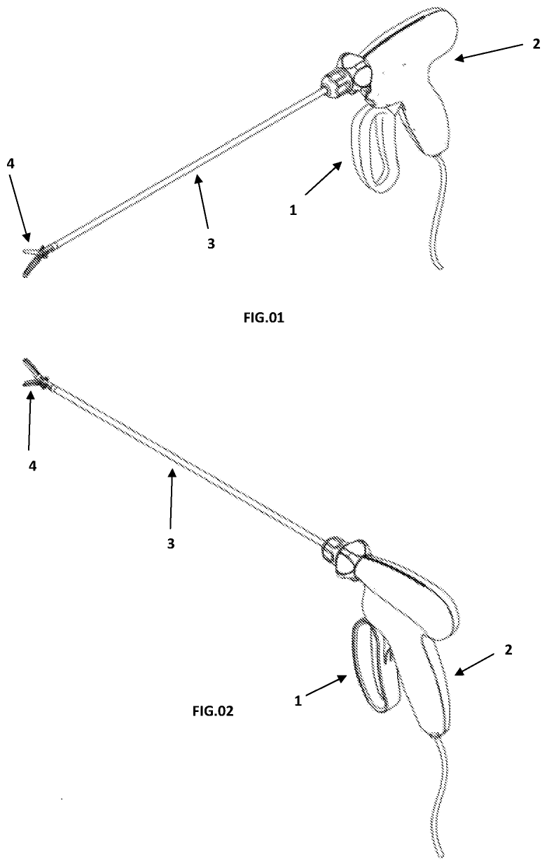

FIG. 02 illustrates the isometric view of the surgical device displaying the handle 2, a trigger 1, outer tube 3 and jaws 4 of the instrument.

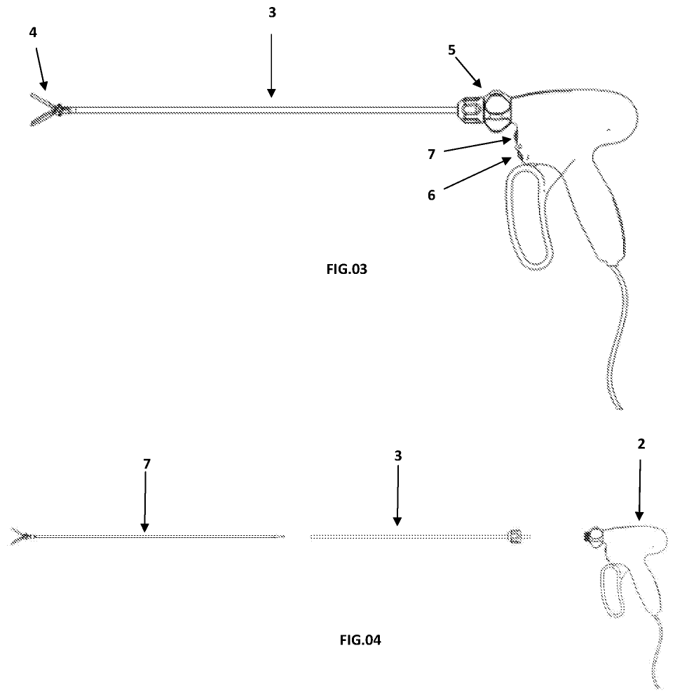

FIG. 03 illustrates the front view of the surgical device displaying a knob 5 for rotating the outer tube 3 and for rotating the jaws 4 as per the requirement of the surgeon so that he can target the precise tissue and avoid unwanted tissue burns. There are two buttons 6 and 7′ that are located on the handle where one button is used for sealing and the other button is used for cutting of the tissue and/or vessel by the surgeon when the trigger 1 is pressed by the surgeon for closing the jaws 4.

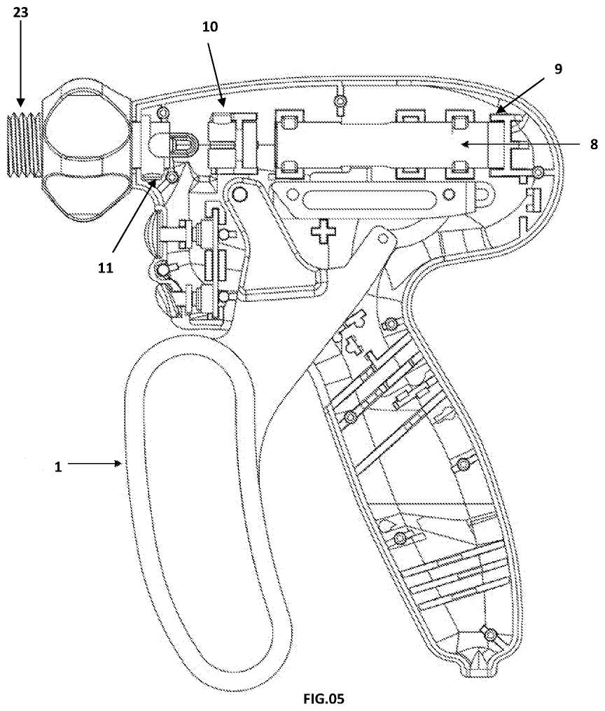

FIG. 04 illustrates the reusable outer tube 3, disposable or reusable jaw insert 7, and reusable handle 2. The handle 2 and outer tube 3 may also be disposed as per use. The jaw insert and the outer tube can be disassembled or detached for cleaning and autoclaving purpose. Old jaw insert can be replaced with the new one if distorted or damaged due to multiple use.

FIG. 05 illustrates the handle showing the threaded joint 23 from which the outer tube 3 and the jaw insert 7 can be detached, for cleaning the jaws for reusing it or for the purpose of changing the jaws to another type of jaw preferred by the surgeon.

Jaw of another shape can be used which can be straight, round, curved or any other shape without any limitation.

FIG. 05 further illustrates the sectional view displaying the internal parts of the device wherein the handle 2 displays an at least one enclosed part 8 which is a jaw driving mechanism which opens and closes the jaws on pressing the trigger 1.

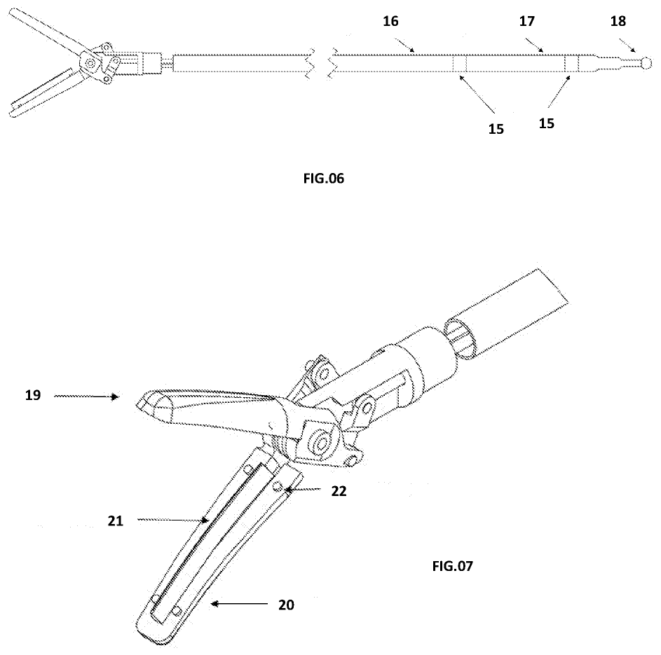

FIG. 6 shows that first contact pole 9 causes electrical conduction on first insert contact 18 of the jaw insert 7, second contact pole 10 causes electrical conduction on second insert contact 17 of the jaw insert 7, third contact pole 11 causes electrical conduction on third insert contact 16 of the jaw insert 7. There are insulators 15 separating the first contact pole, second contact pole, third contact pole. There can be more than three contact poles to achieve various surgical effects of cutting and coagulation

The first and second contact pole causes sealing of the tissue and/or vessel while the third contact pole causes cutting effect.

Minimum two contact poles needed to deliver bipolar energy delivery path. This device has 3 poles and can be increased by adding more number of poles. Various effects can be created by multiple poles or change effect of device.

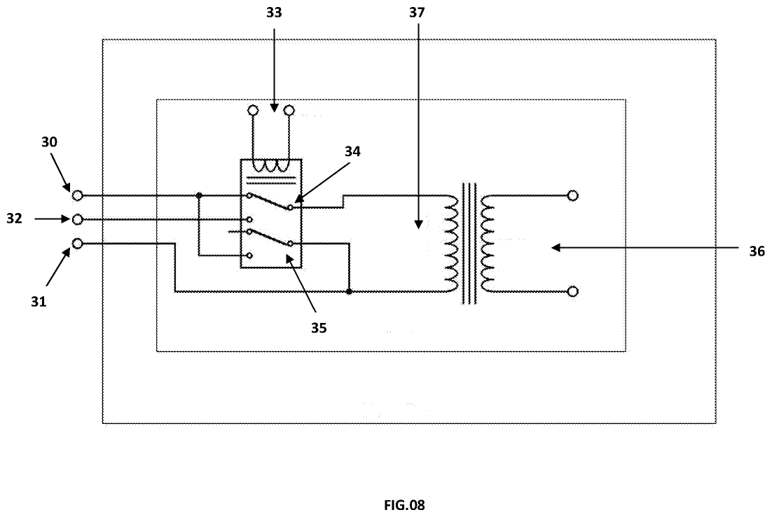

FIG. 7 illustrates the upper jaw 19 and the lower jaw 20 that are used for sealing of the tissue and/or vessel while the cutter 21 is used for cutting. Ceramic spacers 22 are provided inside the jaws to allow spacing when the jaws are closed.

Alternatively the jaws 19 and 20 can be used for sealing without the cutter 21 in between. Alternatively only the cutter 21 can be used for cutting.

FIG. 8 illustrates the generator showing the switching circuit. The circuit shows the isolated output current terminals 36 that provide the bipolar current 37. There is a relay 33. The current switches in mode from S1 (Switch 1, 34) to S2 (Switch 2, 35) and follows a conductive pathway to P1 towards wire (30) and to P2 towards wire (31) and towards wire (32) at cutting element 21.

In an embodiment, the jaws/electrode can have non sticky/superconductive coatings or can be made of superconductive materials or have special chemical coatings for improved results. Electrodes can be made up of nonconductive material with conductive coating on the surface The device can be used for variety of laparoscopic surgical procedures without limitation.

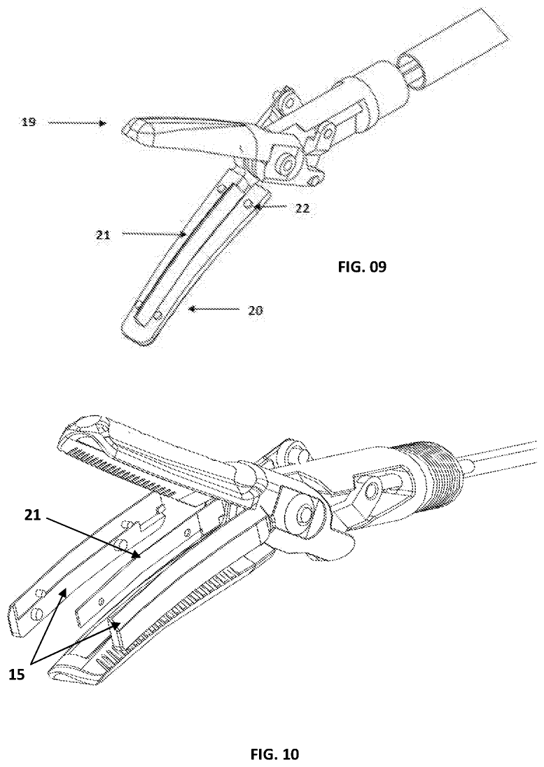

FIG. 09 illustrates a cutter surrounded by insulation to isolate it from the lower jaw.

FIG. 10 illustrates a cutter with an insulation split in two halves.

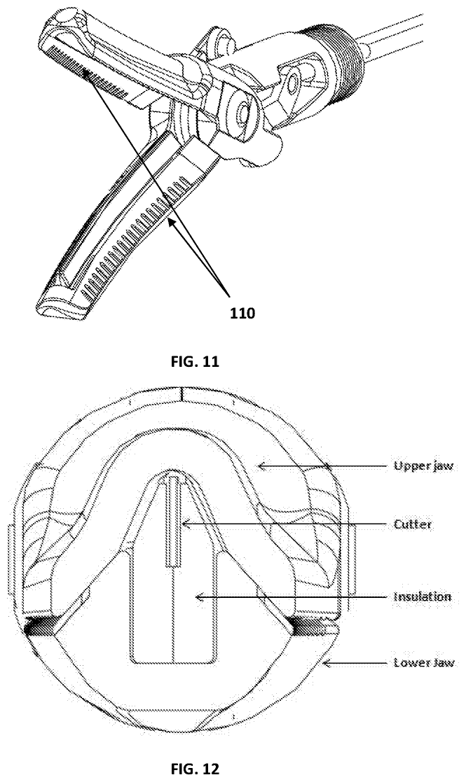

FIG. 11 illustrates jaws having serrations (110) to have better grip over a tissue.

FIG. 12 illustrates a cross section view of jaw assembly having minimized wall thickness and increased surface area to dissipate heat faster which directly reduces the thermal spread on the tissue.

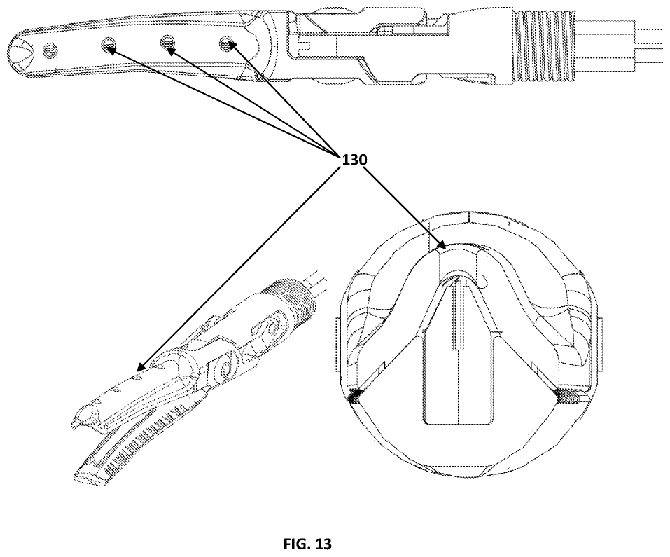

FIG. 13 illustrates a plurality of holes (130) provided on upper jaw (19) in order to release trapped vapors generated during sealing or cutting process. In an embodiment, holes can be given on the upper jaw in order to release trapped vapors generated during sealing or cutting process. Releasing the vapors improves sealing quality.

1-6. (canceled)

7. A bipolar surgical device, comprising:
a jaw insert with jaws connected to an outer tube,
said jaw insert and said outer tube are connected to a handle having at least one enclosed part operating as a jaw driving mechanism,
said jaw driving mechanism operatively coupled to a trigger such that said jaws close when said trigger is pressed and said jaws open when said trigger is not pressed, and
said handle having two buttons, where a first button is used for sealing tissue and/or vessel when pressed, and said second button is used for cutting of the tissue and/or the vessel when pressed,
wherein said jaws comprises at least an upper jaw with a plurality of holes.

8. The bipolar surgical device of claim 7, wherein the jaw insert can be cleaned and/or autoclaved after it is disassembled from the outer tube or it can be replaced.

9. The bipolar surgical device as claimed in claim 7, wherein the outer tube can be disassembled and can be cleaned and/or autoclaved after it is disassembled and can be reused or it can be disposed.

10. The bipolar surgical device as claimed in claim 7, wherein said handle includes a first contact pole, a second contact pole, and a third contact pole.

11. The bipolar surgical device as claimed in claim 10, wherein the first contact pole causes an electrical conduction on a first insert contact of the jaw insert, the second contact pole causes an electrical conduction on a second insert contact of the jaw insert, and the third contact pole causes an electrical conduction on the third insert contact of the jaw insert.

12. The bipolar surgical device as claimed in claim 10, wherein the first contact pole and the second contact pole causes sealing of the tissue and/or vessel while the third contact pole causes the cutting effect.

13. The bipolar surgical device as claimed in claim 11, wherein said first insert contact, said second insert contact and said third insert contact are separated by insulators.

14. The bipolar surgical device as claimed in claim 7, wherein said handle includes threaded joint in which said outer tube and said jaw insert are detachably coupled to said handle.

15. The bipolar surgical device as claimed in claim 7, wherein said jaws comprises the upper jaw having the plurality of holes and a lower jaw separated by spacers allowing space between said upper jaw and said lower jaw when the jaws are closed, wherein said upper jaw and said lower jaw are used for sealing of the tissue and/or vessel, and wherein said plurality of holes enables to release trapped vapors generated during sealing or cutting of the tissue and/or vessel.

16. The bipolar surgical device as claimed in claim 15, wherein said upper jaw or said lower jaw includes a cutter used for cutting the tissue and/or vessel.
